(19) United States
(12) Patent Application Publication
(10) Pub. No.: US 2006/0135613 A1
(43) Pub. Date: Jun. 22, 2006

(54) Carboxamides derivatives

(75) Inventors: Makoto Shimazaki, Soraku-gun (JP); Osamu Sakurai, Otokuni-gun (JP); Kanako Hirai, Sitama-shi (JP); Klaus Urbahns, Lund (SE); Noriyuki Yamamoto, Higashiosaka-shi (JP); Satoru Yoshikawa, Kamakura-shi (JP); Masaomi Umeda, Ritto-shi (JP); Masaomi Tajimi, Chita-shi (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/517,646

(22) PCT Filed: Jun. 12, 2003
(86) PCT No.: PCT/EP03/06168

§ 371 Date: Jan. 27, 2006

(30) Foreign Application Priority Data
Jun. 13, 2002 (GB) 0213598.6

(51) Int. Cl. A61K 31/198; A61K 31/195; C07C 237/38

(52) U.S. Cl. 514/563; 562/450

(57) ABSTRACT

The present invention relates to carboxamides which are useful as an active ingredient of pharmaceutical preparations. The carboxamides of the present invention have IP receptor antagonistic activity, and can be used for the prophylaxis and treatment of diseases associated with IP receptor antagonistic activity. Such diseases include urological diseases or disorder as follows: bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benighn prostatic hypertrophy (BPH), prostatitis, urinary frequency, nocturia, urinary urgency, pelvic hypersensitivity, urethritis, pelvic pain syndrome, prostatodynia, cystitis, or idiophatic bladder hypersensitivity. The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; and inflammation, since the diseases are alleviated by treatment with an IP receptor antagonist.

### DETAILED DESCRIPTION OF INVENTION

1. Technical Field

The present invention relates to a carboxamide derivatives which are useful as an active ingredient of pharmaceutical preparations. The carboxamides of the present invention have IP receptor antagonistic activity, and can be used for the prophylaxis and treatment of diseases associated with IP receptor antagonistic activity.

More specifically, the carboxamide derivatives of the present invention are useful for treatment and prophylaxis of urological diseases or disorders.

The compounds of the present invention are also useful for treatment of pain; hypotension; hemophilia and hemorrhage; inflammation; respiratory states from allegies or asthma, since the disease also is alleviated by treatment with an IP receptor antagonist.

2. Background Art

Prostaglandins (or prostanoids, PGs) are a group of bioactive lipid mediators generated from membrane phospholipids. They are formed from 20-carbon essential fatty acids containing 3, 4, or 5 double bonds, and carry a cyclopentane ring. They are divided into 6 main classes (D, B, F, G, H or I) by the cyclopentane ring structure. The main classes are further subdivided by subscripts 1, 2, or 3, reflecting their fatty acid precursors. PGI2 is a member of prostanoids, and it has a double ring structure and is derived from arachidonic acid. The receptor for PGI2 is a seven transmembrane G-protein coupled receptor, called IP receptor. IP receptor couples at least to Gs-type G-protein, and activates adenylate cyclase and phospholipase C. The expression of IP is demonstrated in aorta, coronary/pulmonary/cerebral arteries, platelets, lung, and dorsal root ganglions in addition to several other tissues.

One of the well-known actions of PGI2 on blood vessels is to cause vasodilation and hypotension. Especially in septic shock, PGI2 is produced and participate in the induction of systemic hypotension (G. D. Bottoms et al, Am J Vet Res 1982, 43(6), 999-1002). Therefore, IP receptor antagonists may prevent hypotension associated with septic shock.

Another well-known action of PGI2 on platelets is to suppress aggregation. In the IP receptor knock out mice, FeCl₃-induced thrombosis formation was enhanced in comparison with that in wild type mice (T. Murata et al, Nature 1997, 388, 678-682.), confirming the involvement of IP receptor in the platelet inhibition. Therefore, IP receptor antagonists may enhance the platelet activation and suppress excessive bleeding such as, but not limited to, hemophilia and hemorrhage.

PGI2 also participate in the inflammation. In the inflamed tissue, various inflammatory mediators, including prostaglandins, are produced. PGI2 is also generated and induces vasodilation to increase blood flow. This enhances vascular permeability, edema formation and leukocyte inflammation in the inflamed region (T. Murata et al, Nature 1997, 388, 678-682.). Therefore, DP receptor antagonists may be efficacious for the treatment of inflammation.

PGI2 may be involved in the pathogenesis of respiratory allergy or asthma. It is spontaneously generated and the major prostaglandin in human lung, and the appropriate antigen challenge increases PGI2 production (E. S. Schulman et al, J Appl Physiol 1982, 53(3), 589-595.). Therefore, IP receptor antagonists may have a utility for the treatment of those respiratory diseases.

In addition, an important role of IP receptor in the induction of hyperalgesia has been clearly shown by IP receptor knockout mice (T. Murata et al., Nature 1997, 388, 678-682.). Injection of acetic acid into the peritoneal cavity induced production of PGI2. This PGI2 is considered to bind to IP receptor on sensory neurons. As IP receptor couples to the activation of both adenylate cyclase and phospholipase C, cAMP-dependent protein kinase (PKA) and protein kinase C (PKC) are activated PKA and PKC are known to modulate ion channels on sensory neurons such as VR1, P2X3, and TTX-R. As a result, PGI2 sensitizes sensory neurons to enhance the release of neurotransmitters. Hence, acetic acid injection induces nociceptive response (writhing) in mice. This acetic acid-induced writhing was greatly reduced in IP receptor-null mice as the same level as indomethacin-treated wild type mice. Several other in vivo hyperalgesia studies in rodents and in vitro studies further support that PGI2 plays a major role in the induction of hyperalgesia and that PGI2 acts as important modulator of sensory neurons (K. Bley et al, Trends in Pharmacological Sciences 1998, 19(4), 141-147.). Therefore, IP receptor antagonists may be useful for the treatment of pain.

Sensory neurons play very important roles not only in the pain sensation but also in the sensation of bladder distension. In normal subjects, A-delta sensory fibers are considered to play a major role to sense the bladder distention. However, in disease conditions of overactive bladder by, but not limited to, spinal cord injury, cystitis, Parkinson's disease, multiple sclerosis, previous cerebrovascular accident, and bladder outlet obstruction (BOO) caused by benign prostate hyperplasia (BPH), the sensitivity of C-fiber sensory neurons is upregulated and they contribute to the induction of the lower urinary tract symptoms. Treatment of overactive bladder patients with intravesical injection of capsaicin or its potent analog, resiniferatoxin, both of which desensitize VR1-positive C-fiber afferent neurons innervating the bladder, has been shown to be efficacious in several clinical trials (C. Silva et al, Bur Urol. 2000, 38(4), 444-452.). Therefore, C-fiber sensory neurons play an important role in the pathology of overactive bladder. PGI2 is generated locally in the bladder and it is the major prostaglandin released from the human bladder. In a rabbit BOO model, a stable metabolite of PGI2 was reported to be increased in BOO bladder (J M. Masick et al, Prostaglandins Other Lipid Mediat. 2001, 66(3), 211-219.). Hence, PGI2 from disease bladder sensitizes C-fiber sensory neurons, and as a result, it may induce symptoms of overactive bladder. Therefore, antagonists of IP receptor are expected to be useful in the treatment of overactive bladder and related urinary disorders.

EP-A-407 200 discloses antihyperlipidemics agents represented by the general formula: 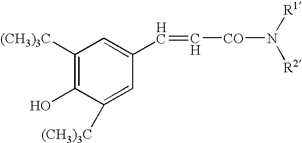
wherein
R^{1′} and R^{2′} are defined in the application.

DE-A-2328391 discloses compounds that are useful for the treatment of heart diseases represented by the general formula:

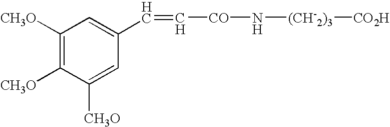

However, none of the references and other reference discloses carboxamides derivatives having IP receptor antagonistic activity.

The development of a compound which has effective IP receptor antagonistic activity and can be used for the prophylaxis and treatment of diseases associated with IP receptor antagonistic activity has been desired.

### SUMMARY OF THE INVENTION

As the result of extensive studies on chemical modification of carboxamides derivatives, the present inventors have found that the compounds of the structure related to the present invention have unexpectedly excellent IP receptor antagonistic activity. The present invention has been accomplished based on these findings.

This invention is to provide a novel carboxamide derivative of the formula (I), its tautomeric or stereoisomeric form, or a salt thereof: 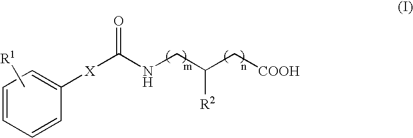
wherein
m and n independently represent an integer from 0 to 2;
—X— represents —CH₂—CH₂—, —CH═CH—, or —C≡C—;
R¹ represents —OR¹¹, —SR¹¹, —SOR¹¹, —SO₂R¹¹, —NR¹²R¹³, or —CHR¹⁴R¹⁵,
   wherein
   R¹¹ represents (C₂₋₆)alkenyl optionally substituted by aryl or heteroaryl, (C₂₋₆)alkyl optionally substituted by aryl or heteroaryl, or (C₁₋₆) alkyl optionally substituted by aryl or heteroaryl,
   R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl,
   or
   R¹² and R¹³ together with the nitrogen atom to which they are attached, form a 5-7 membered saturated hetero ring optionally interrupted by O or NH;
   R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆) alkyl optionally substituted by aryl or heteroaryl, or (C₁₋₆) alkoxy optionally substituted by aryl or heteroaryl,
   or
   R¹⁴ and R¹⁵ together with the CH to which they are attached, form a (C₃₋₈)cycloalkyl optionally interrupted by NH, or O, or a phenyl optionally substituted by hydroxy, halogen or (C₁₋₆) alkyl; and
R² represents hydrogen, cyano, (C₁₋₆) alkoxy, (C₂₋₆)alkenyl, (C₂₋₆)alkynyl, (C₃₋₇)cycloalkyl, or (C₁₋₆) alkyl optionally substituted by amino, (C₁₋₆)alkylamino, or phenyl.

The compounds of the present invention surprisingly show excellent IP receptor antagonistic activity. They are, therefore, suitable for the production of medicament or medical composition, which may be useful for diseases, is alleviated by treatment with an IP receptor antagonist.

More specifically, since the carboxamides derivatives of the present invention antagonize IP receptor, they are useful for treatment and prophylaxis of urological diseases or disorder.

The compounds of the present invention are also useful for treatment of urological diseases or disorders. Such diseases or disorders include bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benigin prostatic hypertrophy (BPH), prostatitis, urinary frequency, nocturia, urinary urgency, pelvic hypersensitivity, urethritis, pelvic pain syndrome, prostatodynia, cystitis, or idiophatic bladder hypersensitivity.

The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; inflammation; respiratory states from allegies or asthma, since the diseases which are alleviated by treatment with IP receptor antagonist.

In another embodiment, the present invention provides a carboxamide derivative of the formula (I′), its tautomeric or stereoisomeric form, or a salt thereof: 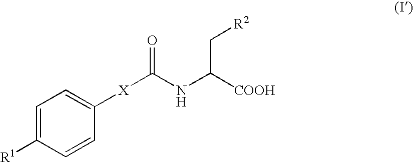
wherein
—X— represents —CH₂—CH₂—, —CH═CH—, or —C≡C—;
R¹ represents —OR¹¹, —SR¹¹, —SOR¹¹, —SO₂R¹¹, —NR¹²R¹³, or —CHR¹⁴R¹⁵,
   wherein
   R¹¹ represents (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl;
   R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl,
   or
   R¹² and R¹³ together with the nitrogen atom to which they are attached, form a 5-7 membered saturated hetero ring optionally interrupted by O or NH;
   R¹⁴ and R¹⁵ independently represent hydrogen, (C₂₋₆)alkenyl optionally substituted by aryl or heteroaryl, (C₂₋₆)alkenyl optionally substituted by aryl or heteroaryl, (C₁₋₆) alkyl optionally substituted by aryl or heteroaryl, or (C₁₋₆) alkoxy optionally substituted by aryl or heteroaryl,
   or
   R¹⁴ and R¹⁵ together with the CH to which they are attached, form a (C₃₋₈)cycloalkyl optionally interrupted by NH, or O, or a phenyl optionally substituted by hydroxy, halogen or (C₁₋₆) alkyl;
R² represents hydrogen, cyano, (C₁₋₆) alkoxy, (C₂₋₆)alkenyl, (C₂₋₆)alkyl, (C₃₋₇)cycloakyl, or (C₁₋₆) alkyl optionally substituted by ammo, (C₁₋₆)alkylamino, or phenyl.

Yet another embodiment of the compounds of formula (a) or (I′) are those wherein:
R¹ represents —OR¹¹, —SR¹¹, —SOR¹¹, SO₂R¹¹, NR¹²R¹³, or —CHR¹⁴R¹⁵,
wherein
   R¹¹ represents (C₂₋₆)alkenyl substituted by aryl or heteroaryl, (C₂₋₆)alkynyl substituted by aryl or heteroaryl, or (C₁₋₆) alkyl substituted by aryl or heteroaryl;
   R¹² and R¹³ independently represent (C₂₋₆)alkenyl substituted by aryl or heteroaryl, (C₂₋₆)alkynyl substituted by aryl or heteroaryl, or (C₁₋₆) alkyl substituted by aryl or heteroaryl;
   R¹⁴ and R¹⁵ independently represent (C₂₋₆)alkenyl substituted by aryl or heteroaryl, (C₂₋₆)alkynyl substituted by aryl or heteroaryl, (C₁₋₆) alkyl substituted by aryl or heteroaryl, or (C₁₋₆) alkoxy substituted by aryl or heteroaryl.

Another embodiment of the compounds of formula (I) or (I′) are those wherein:
R¹ represents phenoxy(C₁₋₆)alkyl, phenoxy(C₁₋₆)alkenyl, phenoxy(C₁₋₆) alkynyl, or phenyl(C₁₋₆)alkoxy.

Further embodiment of the compounds of formula (I) or (I′) are those

wherein

R² represents phenyl (C₁₋₆)alkyl.

Yet further embodiment of the compounds of formula (I) or (I′) are those Wherein
R² represents benzyl.

Further, the present invention provides a medicament which includes one of the compounds described above and optionally pharmaceutically acceptable excipients.

The Alkyl per se and “alk” and “alkyl” in alkoxy, alkanoyl, alkylamino, alkyl-aminocarbonyl, alkylaminosulphonyl, alkylsulphonylamino, alkoxycarbonyl, alkoxy-carbonylamino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Aryl per se represents a mono- to tricyclic aromatic carbocyclic radical having generally 6 to 14 carbon atoms, illustratively and preferably representing phenyl, naphthyl and phenanthrenyl.

Heteroaryl per se represents an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Hetero ring (heterocyclyl) per se represents a mono- or polycyclic, preferably mono- or bicyclic, nonaromatic heterocyclic radical having generally 4 to 10 and preferably 5 to 8 ring atoms and up to 3 and preferably up to 2 hetero atoms and/or hetero groups selected from the group consisting of N, O, S, SO and SO₂. The heterocyclyl radicals can be saturated or partially unsaturated Preference is given to 5- to 8-membered monocyclic saturated heterocyclyl radicals having up to two hetero atoms selected from the group consisting of O, N and S, such as illustratively and preferably tetrahydrofurn-2-yl, pyrrolidin-2-yl, pyrrolidin-3-yl, pyrrolinyl, piperidinyl, morpholinyl, perhydroazepinyl.

### EMBODIMENT OF THE INVENTION

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the methods [A] below.

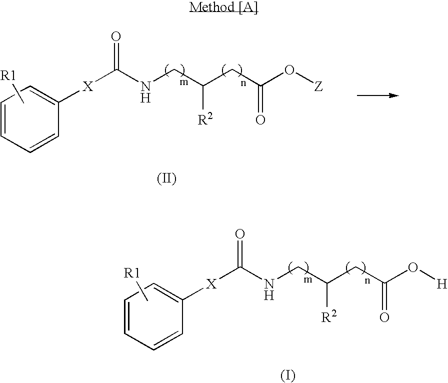

The compound of the formula (I) (wherein R¹, R², X, m, and n are the same as defined above, and Z represents C₁₋₆ alkyl) or a salt thereof can be obtained by the hydrolysis of the starting material of formula (II).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol ethanol, 1-propanol, isopropanol and tert-butanol, water, and others. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

The reaction can be advantageously carried out in the presence of a base including, for instance, an alkali metal alkoxide such as sodium methoxide, sodium ethoxide and potassium tert-butoxide; alkali metal hydroxide such as sodium hydroxide, lithium hydroxide and potassium hydroxide; and others.

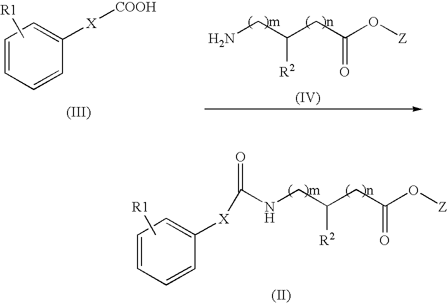

The compound of formula (II) (wherein R¹, R², X, Z, m and n are the same as defined above) can be prepared by the reaction of compound (III) with amine (IV).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); and others. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 24 hours.

The reaction may be carried out using coupling agent including, for instance, carbodiimides such as N,N-dicyclohexylcarbodiimide and 1-(3-dimethylaminopropyl)-3-ethylcarbodiimide, and others.

The compound (III) and amine (IV) can be commercially available or can be prepared by the use of known techniques.

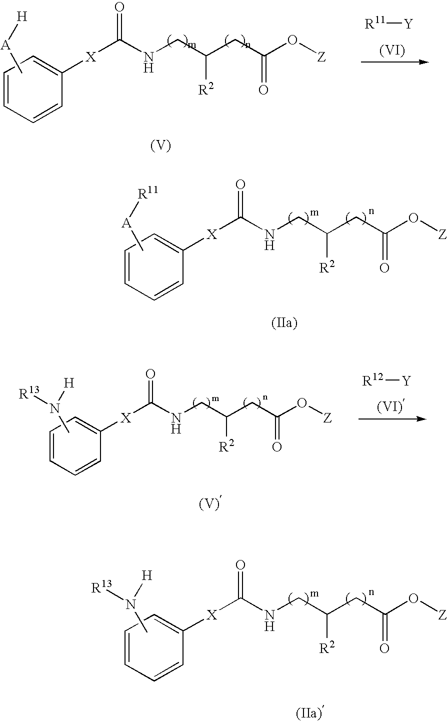

Alternatively, the compound of formula (IIa) or (IIa)′ (Wherein R², R¹¹, R¹², R¹³, X, Z, m and n are the same as defined above and A represents O or S) can be prepared by the reaction of compound (V) or (V)′ with reagents (VI) or (VI)′, respectively (wherein R¹¹ and R¹² are the same as defined above and Y represents a leaving group, such as halogen e.g., chlorine, bromine, or iodine).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); ketones such as acetone; alcohols such as methanol, ethanol, 1-propanol, isopropanol and tert-butanol, and others. Optionally, two or more of the solvents selected from the listed above can be mixed and used.

The reaction temperature can be optionally set depending on the compounds to be reacted. The reaction temperature is usually, but not limited to, about 0° C. to 100° C. The reaction may be conducted for, usually, 30 minutes to 48 hours and preferably 1 to 24 hours.

The reaction can be advantageously carried out in the presence of a base including, for instance, an alkali metal hydride such as sodium hydride or potassium hydride; alkali metal alkoxide such as sodium methoxide, sodium ethoxide and potassium tert-butoxide; alkali metal hydroxide such as sodium hydroxide and potassium hydroxide; alkali metal carbonates such as sodium carbonate and potassium carbonate; alkali metal hydrogen carbonates such as sodium hydrogen carbonate and potassium hydrogen carbonate; alkaline earth metal alkoxides such as magnesium ethoxide; organic amines such as pyridine, triethylamine and N,N-diisopropylethylamine, dimethylaniline, diethylaniline and others.

The compound (V) or (V)′ can be commercially available or can be prepared by either the use of the similar procedure for the preparation of the compound of formula (II) or known techniques. The compound (VI) or (VI)′ can be commercially available or can be prepared by the use of known techniques.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, successively.

Acids to form salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenylsulfonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassium carbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills, powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol calcium carbonate, calcium phosphate, calcium sulfate, methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described in detail below in the form of examples, but they should by no means be construed as defining the metes and bounds of the present invention.

In the examples below, all quantitative data, if not stated otherwise, relate to percentages by weight.

Melting points are uncorrected. Liquid Chromatography-Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. Mass spectra were obtained using electrospray (ES) ionization techniques (micromass Platform LC). TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Tokyo kasei kogyo Co., Ltd., Nacalsi tesque, Inc., Watanabe Chemical Ind. Ltd., Maybridge plc, Lancaster Synthesis Ltd., Merck KgaA, Kanto Chemical Co., Ltd.

The effect of the present compounds were examined by the following assays and pharmacological tests.

[Measurement of the [³H]-Iloprost Binding to HEL Cells] (Assay 1)

A human erythloleukemia cell line, HEL 92.1.7, was purchased from American Type Culture Correction and maintained in RPMI-1640 medium (Gibco BRL) supplemented with 10% fetal calf serum (FCS), 2 mM glutamine, 4.5 g/L glucose, 10 mM Hepes, 1 mM sodium pyruvate, 100 U/ml penicillin and 100 μg/ml streptomycin in a humidified 5% CO₂ atmosphere at 37° C. Cells were collected with centrifugation and washed with binding assay buffer (BAB: 50 mM Tris-HCl, 5 in M MgCl₂ (pH 7.5)). Cells were suspended at the density of 6.25×10⁶ cells/ml in BAB, and one million cells in 160 μl aliquot of cell suspension were put in a well of 96 well plate (Falcon). Then, 20 μl of compound solution, 100 μM of iloprost (for non-specific binding), or buffer alone (total binding), diluted with 1% DMSO in BAB was added. Finally, another 20 μl containing [³H]-iloprost (0.02 μCi, 0.5-1 pmol) in BAB was added and incubated at room temperature for 30 min with a gentle shaking. Cell suspension was then transferred to a well of MultiScreen plate with GF/C glass filters (Millipore) to harvest cells. Cells were washed twice with 200 μl of ice-cold BAB and the plate was kept at 55° C. for 30 min to dry filters. The filter in the well was punched out to a counting tube and 2 ml of Ultima Gold XR (Packard) was added. [3H]-radio activity in the filter was measured by a liquid scintillation counter (Beckman).

[Iloprost-Induced cAMP Production Assay in HEL Cells] (Assay 2)

HEL cells were collected with centrifugation and washed with cAMP assay buffer (CAB: Hank's balanced salt solution, 17 mM Hepes, 0.1% bovine serum albumin, 1 mM IBMX, 0.4% DMSO, and 1 mM L-ascorbic acid sodium salt (pH 7.4)). Cells were suspended at the density of 2.5×10⁵ cells/ml in CAB, and twenty thousand cells in 80 μl aliquot of cell suspension were put in a well of 96 well plate (Falcon). Then, 10 μl of compound solution diluted with 1% DMSO in CAB or buffer alone was added. The plate was incubated at 37° C. for 30 min. Then, another 10 μl containing 100 nM iloprost in CAB or buffer alone was added and further incubated at 37° C. for 30 min. cAMP content in the well was measured by a cAMP ELISA kit (Applied Biosystems).

[Measurement of Rhythmic Bladder Contraction in Anesthetized Rats]

(1) Animals

Female Sprague-Dawley rats (200˜250 g/Charles River Japan) were used.

(2) Rhythmic Bladder Contraction in Anesthetized Rats

Rats were anesthetized by intraperitoneal administration of urethane (Sigma) at 1.25 g/kg. The trachea was cannulated with a polyethylene tube (HIBIKI, No. 8) to facilitate respiration; and a cannula (BECTON DICKINSON, PE-50) was placed in the left femoral vein for intravenous administration of testing compounds. The abdomen was opened through a midline incision, and after both ureters were cut, a water-filled baloon (about 1 ml capacity) was inserted through the apex of the bladder dome. The baloon was connected to a pressure transducer onto a polygraph. Rhythmic bladder contraction was elicited by raising up intravesical pressure to approximately 15 cm H₂O. After the rhythmic bladder contraction was stable, a testing compound was administered intravenously. Activity was estimated by measuring disappearance time and amplitude of the rhythmic bladder contraction. The effect on amplitute of bladder contractions was expressed as a percent suppression of the amplitude of those after the disappearance was recovered. Experimental values were expressed as the mean±S.E.M. The testing compounds-mediated inhibition of the rhythmic bladder contraction was evaluated using Student's t-test. A probability level less than 5% was accepted as significant difference.

Results of IP receptor antagonist assay is shown in Examples below. The data corresponds to the compounds as yielded by solid phase synthesis and thus to levels of purity of about 40 to 90%. For practical reasons, the compounds are grouped in three classes of activity as follows:

IC50=A 0.1 μM<B 1 μM<C

The compounds of the present invention also show excellent selectivity, and strong activity in vivo assays.

### Example 1

### (1) 4-Chloromethylbenzyl Alcohol

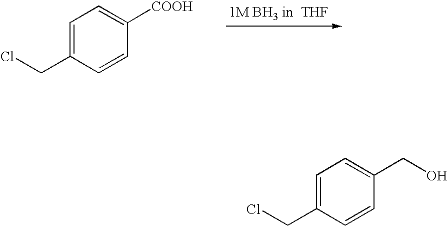

To a solution of 4-chloro-4-toluic acid in tetrahydrofuran (THF, 60 ml) was added 1 M borane THF solution (90 ml). The mixture was stirred at room temperature overnight and quenched by addition of methanol (50 ml). The solvent was evaporated off and the residue was purified by silica gel column chromatography (hexane/ethyl acetate=4/1 to 3/1) to obtain 4-chloromethylbenzyl Alcohol (8.84 g, 96%) as a colorless solid.

### (2) 4-Phenoxymethylbenzyl Alcohol

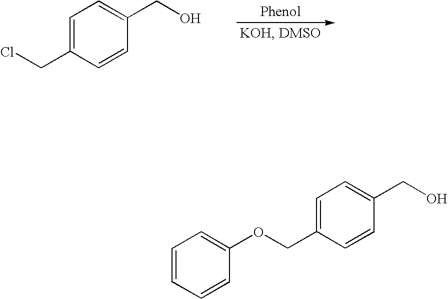

A mixture of 4-chloromethylbenzyl alcohol (0.80 g), phenol (0.48 g), 85% potassium hydroxide (0.76 g) and dimethylsulfoxide (DMSO, 15 ml) was stirred at room temperature overnight and poured into a mixture of water (50 ml) and ethyl acetate (50 ml). The organic layer was washed with brine and dried over sodium sulfate. The solvent was removed off and the residue was purified by silica gel column chromatography (hexane/ethyl acetate=3/1) to obtain 4-phenoxymethylbenzyl alcohol (0.83 g, 76%) as colorless granules.

### (3) 4-Phenoxymethylbenzaldehyde

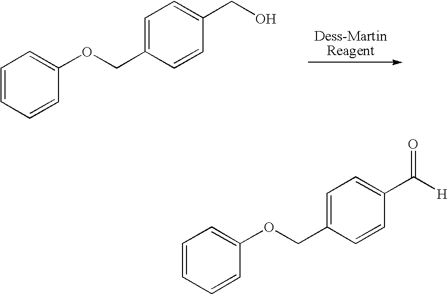

To a solution of Dess-Martin reagent (1.79 g) in dichloromethane (10 ml) was dropwise added a solution of 4-phenoxymethylbenzyl alcohol (0.82 g) at room temperature. The mixture was stirred at room temperature for 30 min and poured into 1N NaOH water solution (30 ml). The organic layer was washed with water and dried over sodium sulfate. The solvent was removed off and the residue was purified by silica gel column chromatography (hexane/ethyl acetate=4/1) to obtain 4-phenoxymethylbenzaldehyde (0.67 g, 81%) as a colorless solid.

(4) tert-Butyl 4-Phenoxymethylcinnamate

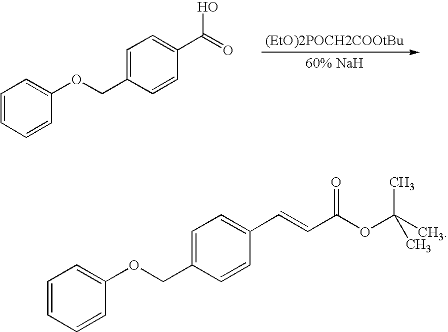

To a solution of tert-butyl diethoxyphosphorylacetate (0.66 g) in THF (10 ml) was added 60% sodium hydride (0.10 g) at 0° C. The mixture was stirred for 1 hr on an ice-water bath and a solution of 4-phenoxymethylbenzaldehyde in THF (1 ml) was added dropwise. The reaction mixture was stirred at room temperature overnight and poured into saturated ammonium chloride water solution (50 ml). The resulting suspension was extracted with ethyl acetate and the organic layer was washed dried over sodium sulfate. The solvent was removed and the residue was purified by silica gel column chromatography (hexane/ethyl acetate 4/1) to obtain tert-butyl 4-phenoxymethylcinnamate (0.73 g, 100%) as a colorless solid.

### (5) N-(4-Phenoxymethylcinnamoyl)phenylalanine Methyl Ester

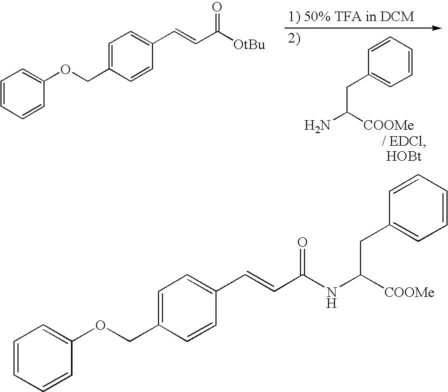

A mixture of tert-butyl 4-phenoxymethylcinnamate (0.20 g), trifluoroacetic acid (TFA, 1 ml) and dichloromethane (1 ml) was allowed to stand for 2.5 hr at room temperature. The solvent was removed in vacuo and the residue was dissolved in N,N-dimethylformamide (DMF, 5 ml). To the solution were added phenylalanine methyl ester (0.15 g), 1-ethyl-3-(3-dimethylaminopropyl)carbodiimide (EDCI, 0.18 g), 1-hydroxybenzotriazole (HOBt, 0.12 g) and triethylamine (0.12 ml). The mixture was stirred at room temperature overnight and poured into a mixture of water (30 ml) and ethyl acetate (20 ml). The organic layer was washed with water and dried over sodium sulfate. The solvent was removed off and the residue was purified by silica gel column chromatography (hexane/ethyl acetate=2/1) to obtain N-(4-phenoxymethylcinnamoyl)phenylalanine methyl ester (0.22 g, 88%) as a colorless solid.

### (6) N-(4-phenoxymethylcinnamoyl)phenylalanine

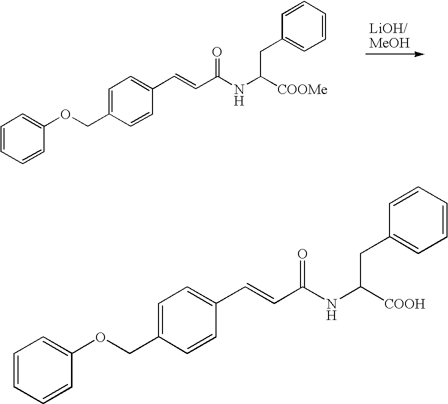

To a solution of N-(4-phenoxymethylcinnamoyl)phenylalanine methyl ester (70 mg) in methanol (2 ml) was added 1N lithium hydroxide water solution (0.2 ml). The mixture was stirred at 50° C. overnight and concentrated in vacuo. The residue was dissolved in water and acidified with 1N hydrochloric acid. The resulting suspension was extracted with ethyl acetate. The organic layer was washed with brine and dried over sodium sulfate. The solvent was removed off and the residue was triturated with diisopropyl ether to obtain N-(4-phenoxymethylcinnamoyl)phenylalanine (59 mg, 86%) as a colorless solid.

mp 205° C.; Calcd [M+1]: 402, Found: m/z 402.

Molecular weight: 401.47

Activity grade assay 2: A

¹H-NMR (500 MHz, DMSO-d6): δ 2.94 (1H, dd, J=14.2, 9.4 Hz), 3.12 (1H, dd, J=13.9, 4.8 Hz), 4.55-4.59 (1H, m), 5.12 (2H, s), 6.70 (1H, d, J=17.0 Hz), 6.94 (1H, t, =7.3 Hz), 7.01 (2H, dd, J=8.5, 0.9 Hz), 7.18-7.31 (6H, m), 7.38 (1H, d, J=15.8 Hz), 7.48 (2H, d, J=8.2 Hz), 7.56 (2H, d, J=8.2 Hz), 8.40 (1H, d, J=8.2 Hz), 12.76 (1H, s).

### Example 2

(1) tert-Butyl 3-(4-Phenoxymethylphenyl)propionate

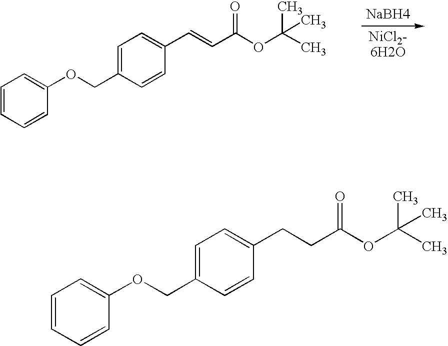

To a mixture of tert-butyl 4-phenoxymethylcinnamate (see: example 1-(4), 0.20 g) and nickel chloride hexahydrate (0.02 g) in methanol (4 ml) was added sodium borontetrahydride (0.05 g) on an ice-water bath. The mixture was stirred at room temperature for 1 hr and quenched with saturated ammonium chloride water solution. The resulting suspension was extracted with ethyl acetate and the organic layer was washed with water and dried over sodium sulfate. The solvent was removed off and the residue was purified by silica gel column chromatography (hexane/ethyl acetate=4/1) to obtain tert-butyl 3-(4-phenoxymethylphenyl)propionate (0.168 g, 840%) as a colorless solid.

### (2) N-[3-(4-Phenoxymethylphenyl)propionyl]phenylalanine

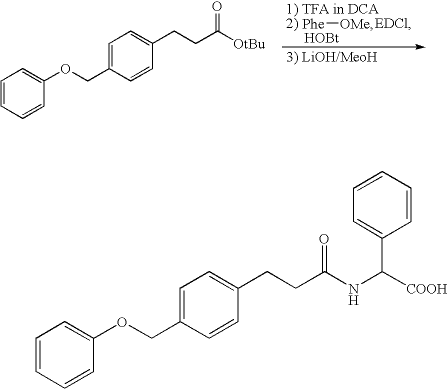

To a solution of tert-butyl 3-(4-phenoxymethylphenyl)propionate (0.10 g) in ethanol (2 ml) was added 1N lithium hydroxide water solution (0.7 ml). The mixture was stirred at 60° C. for 3 hr and concentrated in vacuo. The residue was suspended in a mixture of 1N hydrochloric acid (0.7 ml), water (5 ml) and ethyl acetate (10 ml) and the organic layer was washed with brine and dried over sodium sulfate. The solvent was removed off and the residue was used for the following steps towards N-[3-(4-Phenoxymethylphenyl)propionyl]phenylalanine, according to the procedures for the synthesis of N-(4-phenoxymethylcinnamoyl)phenylalanine (See: example 1-(5) and (6)).

mp 173-174° C.; Calcd [M+1]: 404, Found: m/z 404.

Molecular weight: 403.48

Activity grade assay 2: A

¹H-NMR (500 MHz, DMSO-d6): δ 2.37 (2H, t, J=8.1 Hz), 2.72 (2H, t, J=7.9 Hz), 2.84 (1H, dd, 3=13.9, 9.5 Hz), 3.03 (1H, dd, J=13.9, 5.0 Hz), 4.41-4.45 (1H, m), 5.04 (2H, s), 6.93 (1H, t, 3=7.3 Hz), 6.99 (2H, dd, J=8.9, 1.0 Hz), 7.15 (2H, d, J=8.2 Hz), 7.18-7.20 (3H, m), 7.24-7.31 (5H, m), 8.17 (1H, d, 3=8.2 Hz), 12.66 (1H, bs).

### Example 3

### (1) 1-Iodo-4-(phenoxymethyl)benzene

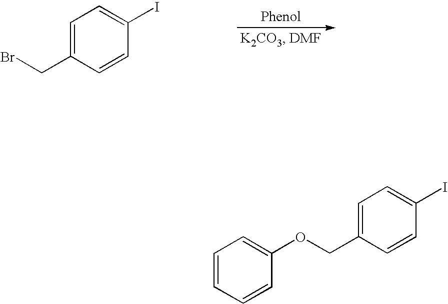

A mixture of 4-iodobenzyl bromide (1 g), phenol (0.286 g), potassium carbonate (0.530 g) and DMF (20 ml) was stirred at room temperature overnight. The volatiles were removed off in vacuo and the residue was suspended in a mixture of ethyl acetate and water. The organic layer was separated to be washed with brine and dried over sodium sulfate. The solvent was removed and the residue was purified by silica gel column chromatography to obtain 1-iodo-(phenoxymethyl)benzene (0.918 g, 93%) as pale yellow flakes.

### (2) Methyl 4-Phenoxymethylphenylpropiolate

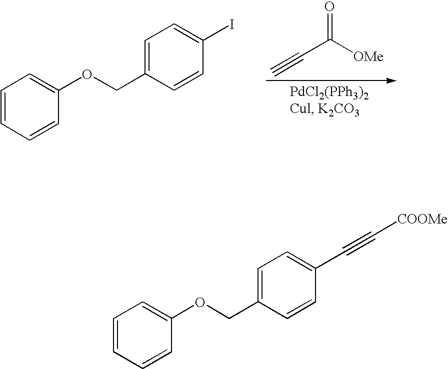

To a solution of 1-iodo-4-(phenoxymethyl)benzene (0.40 g) and methyl propiolate (0.43 g) in THF (8 ml) were added Biskis(triphenylphosphine)palladium dichloride (18 mg), cuprous iodide (10 mg) and potassium carbonate (0.36 g). The mixture was stirred at 80° C. and consentrated in vacuo. The residue was purified by silica gel column chromatography (hexane/ethyl acetate=10/1) to obtain methyl 4-phenoxymethylphenylpropiolate (0.155 g, 45%) as colorless flakes.

### (3) N-(4-Phenoxymethylphenylpropioloyl)phenylalanine

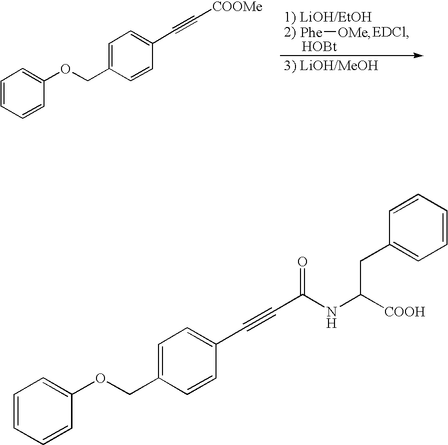

According to the procedure for the synthesis of N-(4-phenoxymethylcinnamoyl)phenylalanine (See: example 1-(5) and (6)) from tert-butyl 4-phenoxymethylcinnamate, N-(4-phenoxymethylphenylpropioloyl)phenylalanine was prepared from 4-phenoxymethylphenylpropiolic acid, which was obtained from the corresponding methyl ester by the hydrolysis with 1N lithium hydroxide in ethanol.

mp 146° C.; Calcd [M+1]: 400, Found: m/z 400.

Molecular weight: 399.44

Activity grade assay 2: A

¹H-NMR (500 MHz, DMSO-d6): δ 2.92 (1H, dd, J 13.8, 10.1 Hz), 3.13 (1H, d, J=13.9, 4.7 Hz), 4.46-4.51 (1H, m), 5.16 (2H, s), 6.95 (1H, t, J=7.3 Hz), 7.01 (2H, d, J=7.9 Hz), 7.20-7.31 (6H, m), 7.52 (2H, d, J=8.2 Hz), 7.59 (2H, d, J=8.2 Hz), 9.14 (1H, d, 3=8.2 Hz), 12.88 (1H, bs).

### Example 4

### (1) 4-Benzyloxybenzaldehyde

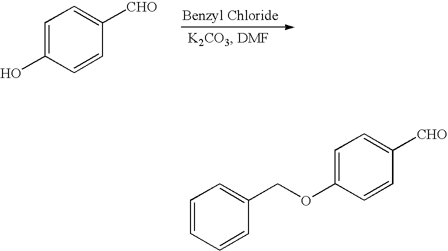

To a solution of 4-hydroxybenzaldehyde (1.00 g) in DMF (30 ml) were added benzyl chloride (1.24 g) and potassium carbonate (1.36 g). The mixture was stirred at room temperature overnight and at 60° C. for 2 hr. The reaction mixture was concentrated in vacuo and the residue was suspended in a mixture of ethyl acetate and water. The organic layer was separated to be washed with brine and dried over magnesium sulfate. The solvent was removed in vacuo and the residue was purified by silica gel column chromatography (hexane/ethyl acetate=2/1) to obtain 4-benzyloxybenzaldehyde (1.82 g, 100%) as a colorless solid.

### (2) N-(4-Benzyloxycinnamoyl)phenylalanine

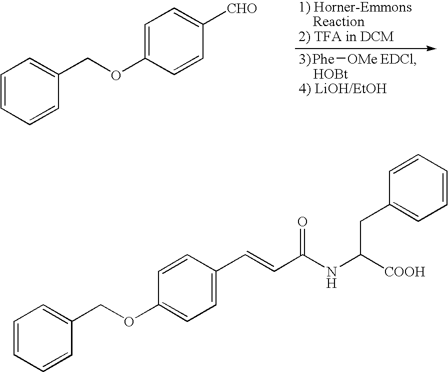

According to the procedures for the synthesis of tert-butyl 4-phenoxymethylcinnamate (See: example 1-(4)), 4-benzyloxybenzaldehyde was subjected to the described Homer-Emmons reaction to obtain tert-butyl 4-benzyloxycinnamate, followed by hydrolysis with lithium hydroxide in ethanol using the procedure described in example 2-(2). Resulting 4-benzyloxycinnamic acid was coupled with phenylalanine methyl ester and hydrolyzed with lithium hydroxide in ethanol to obtain N-(4-benzyloxycinnamoyl)phenylalanine, according to the procedure for the synthesis of N-(4-phenoxymethylcinnamoyl)phenylalanine (See: example 1-(6)).

mp 220° C.; Calcd [M+1]: 402, Found: m/z 402.

Molecular weight: 401.46

Activity grade assay 2: A

¹H-NMR (500 MHz, DMSO-d6): δ 2.92 (1H, dd, J=9.5, 13.9 Hz), 3.11 (1H, dd, J=5.1, 14.2 Hz), 4.55 (1H, m), 5.14 (2H, s), 6.55 (1H, d, J=15.8 Hz), 7.05 (2H, d, J=8.8 Hz), 7.18-7.21 (1H, m), 7.23-7.35 (6H, m), 7.38-7.41 (1H, m), 7.44-7.46 (1H, m), 7.49 (2H, d, J=8.9 Hz), 12.77 (1H, br s).

1. A carboxamide derivative of the formula (I), its tautomeric or stereoisomeric form, or a salt thereof: 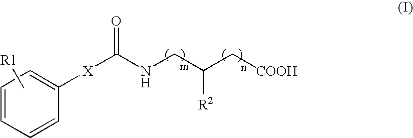
wherein
m and n independently represent an integer from 0 to 2;
—X— represents —CH₂—CH₂—, —CH═CH—, or —C≡C—;
R¹ represents —OR¹¹, —SR¹¹, —SOR¹¹, —SO₂R¹¹, —NR¹²R¹³, or —CHR¹⁴R¹⁵,
wherein
R¹¹ represents (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl;
R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl,
or
R¹² and R¹³ together with the nitrogen atom to which they are attached, form a 5-7 membered saturated hetero ring optionally interrupted by O or NH;
R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆) alkyl optionally substituted by aryl or heteroaryl, or (C₁₋₆) alkoxy optionally substituted by aryl or heteroaryl,
or
R¹⁴ and R¹⁵ together with the CH to which they are attached, form a (C₃₋₈)cycloalkyl optionally interrupted by NH, or O, or a phenyl optionally substituted by hydroxy, halogen or (C₁₋₆) alkyl; and
R² represents hydrogen, cyano, (C₁₋₆) alkoxy, (C₂₋₆)alkenyl, (C₂₋₆)alynyl, (C₃₋₇)cycloalkyl, or (C₁₋₆) alkyl optionally substituted by amino, (C₁₋₆)alkylamino, or phenyl.

2. A carboxamide derivative of the formula (I′), its tautomeric or stereoisomeric form, or a salt thereof: 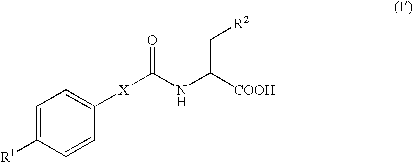
wherein
—X— represents —CH₂—CH₂—, —CH═CH—, or —C≡C—;
R¹ represents —OR¹¹, —SR¹¹, —SOR¹¹, —SO₂R¹¹, —NR¹²R¹³, or —CHR¹⁴R¹⁵,
wherein
R¹¹ represents (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl;
R¹² and R¹³ independently represent hydrogen, (C₂₋₆)alkenyl optionally substituted by aryl or heteroaryl, (C₂₋₆)alkynyl optionally substituted by aryl or heteroaryl, or (C₁₋₆) alkyl optionally substituted by aryl or heteroaryl,
or
R¹² and R¹³ together with the nitrogen atom to which they are attached, form a 5-7 membered saturated hetero ring optionally interrupted by O or NH;
R¹⁴ and R¹⁵ independently represent hydrogen, (C₂₋₆)alkenyl optionally substituted by aryl or heteroaryl, (C₂₋₆)alkynyl optionally substituted by aryl or heteroaryl, (C₁₋₆) alkyl optionally substituted by aryl or heteroaryl, or (C₁₋₆) alkoxy optionally substituted by aryl or heteroaryl,
or
R¹⁴ and R¹⁵ together with the CH to which they are attached, form a (C₃₋₈)cycloalklyl optionally interrupted by NH, or O, or a phenyl optionally substituted by hydroxy, halogen or (C₁₋₆) alkyl; and
R² represents hydrogen, cyano, (C₁₋₆) alkoxy, (C₂₋₆)alkenyl, (C₂₋₆)alkynyl, (C₃₋₇)cycloalkyl, or (C₁₋₆) alkyl optionally substituted by amino, (C₁₋₆)alkylamino, or phenyl.

3. The carboxamide derivative, its tautomeric or stereoisomeric form, or a salt thereof as claimed in claim 1 or 2,
wherein
R¹¹ represents —OR¹¹, —SR¹¹, —SOR¹¹, —SO₂R¹¹, —NR¹²R¹³, or —CHR¹⁴R¹⁵,
wherein
R¹¹ represents (C₂₋₆)alkenyl substituted by aryl or heteroaryl, (C₂₋₆)alkenyl substituted by aryl or heteroaryl, or (C₁₋₆) alkyl substituted by aryl or heteroaryl;
R¹² and R¹³ independently represent (C₂₋₆)alkenyl substituted by aryl or heteroaryl, (C₂₋₆)alkynyl substituted by aryl or heteroaryl, or (C₁₋₆) alkyl substituted by aryl or heteroaryl;
R¹⁴ and R¹⁵ independently represent (C₂₋₆)alkenyl substituted by aryl or heteroaryl, (C₂₋₆alkynyl substituted by aryl or heteroaryl, (C₁₋₆) alkyl substituted by aryl or heteroaryl, or (C₁₋₆) alkoxy substituted by aryl or heteroaryl.

4. The carboxamide derivative, its tautomeric or stereoisomeric form, or a salt thereof as claimed in claim 1 or 2,
wherein
R¹ is phenoxy(C₁₋₆)alkyl, phenoxy(C₁₋₆)alkenyl, phenoxy(C₁₋₆)alkynyl, or phenyl(C₁₋₆)alkoxy.

5. The carboxamide derivative, its tautomeric or stereoisomeric form, or a salt thereof as claimed in claim 1 or 2,
wherein
R² is phenyl (C₁₋₆)alkyl.

6. The carboxamide derivative, its tautomeric or stereoisomeric form, or a salt thereof as claimed in claim 1 or 2,
wherein
R² is benzyl.

7. The carboxamide derivative, its tautomeric or stereoisomeric form, or a salt thereof as claimed in claim 1, wherein said derivative is selected from the group consisting of the following compounds:
N-(4-phenoxymethylcinnamoyl)phenylalanine;
N-[3-(4-Phenoxymethylphenyl)propionyl]phenylalanine;
N-(4-Phenoxymethylphenylpropioloyl)phenylalanine; and
N-(4-Benzyloxycinnamoyl)phenylalanine.

8. A medicament comprising the carboxamide derivative, its tautomeric or stereoisomeric form, or a physiologically acceptable salt thereof as claimed in claim 1 or 2 as an active ingredient.

9. The medicament as claimed in claim 8, further comprising one or more pharmaceutically acceptable excipients.

10. The medicament as claimed in claim 8, wherein the carboxamide derivative, its tautomeric or stereoisomeric form, or a physiologically acceptable salt thereof is an IP receptor antagonist.

11. The medicament as claimed in claim 8 for prophylaxis and/or treatment of urological disorder or disease.

12. The medicament as claimed in claim 8 for prophylaxis and/or treatment of pain.

13. The medicament as claimed in claim 8 for prophylaxis and/or treatment of hypotension.

14. The medicament as claimed in claim 8 for prophylaxis and/or treatment of hemophilia and hemorrhage.

15. The medicament as claimed in claim 8 for prophylaxis and/or treatment of inflammation.

16. Use of compounds according to claims 1 for manufacturing a medicament for the treatment and/or prophylaxis of urological disorders.

18. Process for controlling urological disorders in humans and animals by administration of an IP receptor-antagonisticly effective amount of at least one compound according to claims 1.
